(19) United States
(12) Patent Application Publication
(10) Pub. No.: US 2022/0287661 A1
(43) Pub. Date: Sep. 15, 2022

(54) Bite Tray for Alleviating Dental Discomfort

(71) Applicant: Flavia Sweeps, Houston, TX (US)

(72) Inventor: Flavia Sweeps, Houston, TX (US)

(21) Appl. No.: 17/199,698

(22) Filed: Mar. 12, 2021

(51) Int. Cl. A61B 6/14

(52) U.S. Cl. CPC A61B 6/145

(57) ABSTRACT

A bite tray, system, and method for alleviating dental discomfort includes a first mouth guard section, a second mouth guard section, a hinge section, and a channel. Each of first and second mouth guard sections include a first surface, a second surface, an anterior region and a posterior region. The first mouth guard section and the second mouth guard section are adapted to receive the teeth of a subject. The hinge section interconnects first and second mouth guard sections to allow the anterior region of each to open and close with respect to each other. The channel is formed when the first surface of the first mouth guard section and the first surface of the second mouth guard are brought together. The channel is adapted to receive and hold a bite plate of an intra-oral radiographic device.

### TECHNICAL FIELD

The present disclosure is directed to gloves. More specifically, this disclosure is directed to a safety feature for a glove.

### BACKGROUND

There are several types of intra-oral exams given by dentists.

One type of intra-oral exam is known as a bitewing radiograph. FIG. 1 depicts a bitewing radiograph 110 comprising an intraoral film packet 120 and a wing 130. The intraoral film packet is placed into the mouth of a patient. The image receptor is positioned so as to lie parallel to the bite-platform. The patient bites on the small wing attached to the intraoral film packet to hold the image receptor firmly against upper and lower teeth. The dental practitioner points an X-ray beam-aiming device in the direction of the image receptor to expose the image receptor to X-ray radiation. The image receptor captures the visible part of the upper and lower teeth as well as part of the roots and supporting bone.

Another type of intra-oral exam is known as digital radiography. Digital radiography or x-rays provide accurate images of patient's teeth. Instead of an image receptor, digital radiography uses a sensor placed in or moved around the mouth to get a full view of the teeth and jawbone. FIG. 2 depicts a conventional digital radiograph 210 comprising an image receptor 215 held by an image receptor support and holder 220, a bite block 230, a locator ring 240, a metal arm 250, and an x-ray source (not shown). The receptor 215 is placed into the mouth of a patient. The receptor is positioned so as to lie parallel to the bite-platform. The patient bites on the bite block 230 attached to the image receptor 215 to hold the image receptor firmly against upper and lower teeth. The dental practitioner points an X-ray beam-aiming device in the direction of the image receptor to expose the image receptor to X-ray radiation. The locator ring 240 and metal arm 250 are used by the practitioner as an aiming device to aim the X-ray. The image receptor captures the visible part of the upper and lower teeth as well as part of the roots and supporting bone.

These intra-oral exams given by dentists may cause patient discomfort. Patient discomfort may arise from placement of the intraoral film packet into the mouth of the patient. For, example, in finding the right position the intraoral film packet may be pressed against the gums of the patient. The edges of the intraoral film packets may press against and scrape the roof of the mouth during positioning. Once properly positioned, the intraoral film packet may continue to press against the gums and roof of the mouth of the patient while the X-rays are being taken.

There is a need for an intra-oral exam device that may be placed into, moved about and positioned within the oral cavity of a patient in a way that creates less discomfort to the patient. This disclosure addresses that need.

Unless otherwise indicated herein, the materials described in this section are not prior art to the claims in this application and are not admitted to be prior art by inclusion in this section.

### SUMMARY

A bite tray for alleviating dental discomfort includes a first mouth guard section, a second mouth guard section, a hinge section, and a channel. The first mouth guard section includes a first surface, a second surface, an anterior region and a posterior region. The first mouth guard section is adapted to receive the top teeth of a subject. The second mouth guard section includes a first surface, a second surface, an anterior region and a posterior region. The second mouth guard section is adapted to receive the bottom teeth of a subject. The hinge section includes a superior end and an inferior end. The superior end of the hinge section is connected to the posterior region of the first mouth guard and the interior end of the hinge section is connected to the second mouth guard. The hinge section is adapted to allow the anterior region of the first mouth guard section and the anterior region of the second mouth guard to open and close and the posterior region of the first mouth guard section and the posterior region of the second mouth guard to have limited movement with respect to each other. The channel is formed when the first surface of the first mouth guard section and the first surface of the second mouth guard are brought together. The channel extends along a predetermined length of the first surface of the first mouth guard section and the first surface of the second mouth guard. The channel opens on one side toward the facial direction of a subject and on another side toward the lingual direction of a subject. The channel is adapted to receive and hold a bite plate of an intra-oral radiographic device.

A system for intra-oral radiography includes a bite tray for alleviating dental discomfort and an intra-oral radiographic device. The bite tray includes a first mouth guard section, a second mouth guard section, a hinge section, and a channel. The first mouth guard section includes a first surface, a second surface, an anterior region and a posterior region. The first mouth guard section is adapted to receive the top teeth of a subject. the second mouth guard section includes a first surface, a second surface, an anterior region and a posterior region. The second mouth guard section is adapted to receive the bottom teeth of a subject. The hinge section includes a superior end and an inferior end. The superior end of the hinge section is connected to the posterior region of the first mouth guard and the interior end of the hinge section connected to the second mouth guard. The hinge section is adapted to allow the anterior region of the first mouth guard section and the anterior region of the second mouth guard to open and close and the posterior region of the first mouth guard section and the posterior region of the second mouth guard to have limited movement with respect to each other. The channel is formed when the first surface of the first mouth guard section and the first surface of the second mouth guard are brought together, the channel extending along a predetermined length of the first surface of the first mouth guard section and the first surface of the second mouth guard, the channel opening on one side toward the facial direction of a subject and on another side toward the lingual direction of a subject. The intra-oral radiographic device includes a bite plate and an imaging device. The channel of the bite tray is adapted to receive and hold the bite plate of an intra-oral radiographic device. The imaging device is held against one or more teeth of either or both a left or right teeth set of a dental structure when the channel of the bite tray receives and holds the biteplate of the intra-oral radiographic device.

A method for intra-oral radiography includes the steps of: (1) providing a bite tray for alleviating dental discomfort. The bite tray includes a first mouth guard section, a second mouth guard section, a hinge section, and a channel. The first mouth guard section includes a first surface, a second surface, an anterior region and a posterior region. The first mouth guard section is adapted to receive the top teeth of a subject. The second mouth guard section includes a first surface, a second surface, an anterior region and a posterior region. The second mouth guard section is adapted to receive the bottom teeth of a subject. The hinge section includes a superior end and an inferior end. The superior end of the hinge section is connected to the posterior region of the first mouth guard and the interior end of the hinge section connected to the second mouth guard. The hinge section is adapted to allow the anterior region of the first mouth guard section and the anterior region of the second mouth guard to open and close and the posterior region of the first mouth guard section and the posterior region of the second mouth guard to have limited movement with respect to each other. The channel is formed when the first surface of the first mouth guard section and the first surface of the second mouth guard are brought together, the channel extending along a predetermined length of the first surface of the first mouth guard section and the first surface of the second mouth guard, the channel opening on one side toward the facial direction of a subject and on another side toward the lingual direction of a subject, the channel adapted to receive and hold a bite plate of an intra-oral radiographic device; (2) providing an intra-oral radiographic device including an imaging source, a bite plate and an imaging receptor; (3) receiving and holding the bite plate of an intra-oral radiographic device in the channel of the bite tray; (4) holding the imaging device against one or more teeth of either or both a left or right teeth set of a dental structure when the channel of the bite tray receives and holds the biteplate of the intra-oral radiographic device; (5) aiming the imaging source of the intra-oral radiographic device in the direction of the imaging receptor; and (6) activating the imaging source of the intra-oral radiographic device to cause the imaging receptor to capture an image of the one or more teeth of either or both a left or right teeth set of a dental structure.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1 shows a conventional bitewing radiograph.

FIG. 2 shows a conventional digital radiograph.

FIGS. 3A, 3B (collectively FIG. 3) show a prior art mouth guard for protecting teeth.

FIGS. 4A, 4B (collectively FIG. 4) show an illustrative embodiment bite tray of this disclosure configured for bitewing radiograph.

FIGS. 5A, 5B, 5C (collectively FIG. 5) show illustrative embodiment of channels formed in a bite tray according to this disclosure.

FIGS. 6A, 6B (collectively FIG. 6) show an illustrative embodiment of a bite tray of this disclosure configured for bitewing or digital radiograph.

FIG. 7 depicts a dental anatomy.

FIGS. 8A, 8B, 8C (collectively FIG. 8) show an illustrative embodiment of a bite tray according to this disclosure for bitewing radiograph.

FIGS. 9A, 9B, 9C, 9D (collectively FIG. 9) show an illustrative embodiment of a bite tray according to this disclosure for bitewing radiograph.

FIGS. 10A, 10B, 10C (collectively FIG. 10) show an illustrative embodiment of a bite tray according to this disclosure for digital radiograph.

FIGS. 11A, 11B, 11C, 11D (collectively FIG. 11) show an illustrative embodiment of a bite tray according to this disclosure for digital radiograph.

FIG. 12 shows a method for intra-oral radiography according to this disclosure.

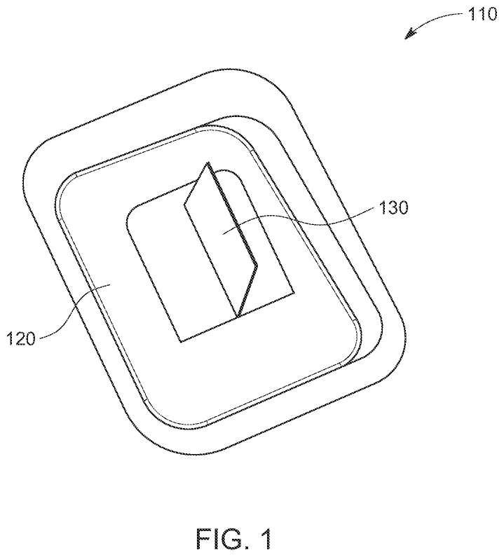

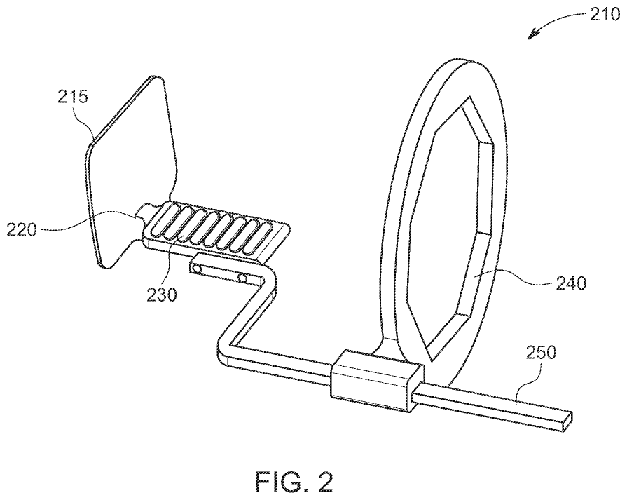

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein. Broadly speaking, a bite tray, system, and method for alleviating dental discomfort includes a first mouth guard section, a second mouth guard section, a hinge section, and a channel. Each of first and second mouth guard sections include a first surface, a second surface, an anterior region and a posterior region. The first mouth guard section and the second mouth guard section are adapted to receive the teeth of a subject. The hinge section interconnects first and second mouth guard sections to allow the anterior region of each to open and close with respect to each other. The channel is formed when the first surface of the first mouth guard section and the first surface of the second mouth guard are brought together. The channel is adapted to receive and hold a bite plate of an intra-oral radiographic device.

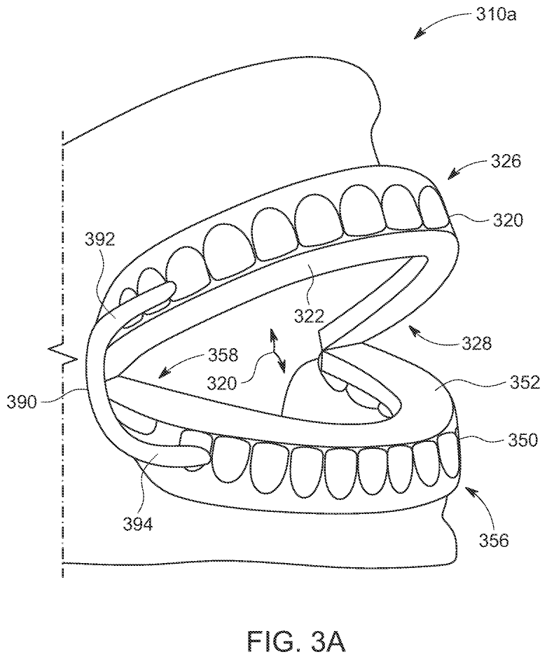

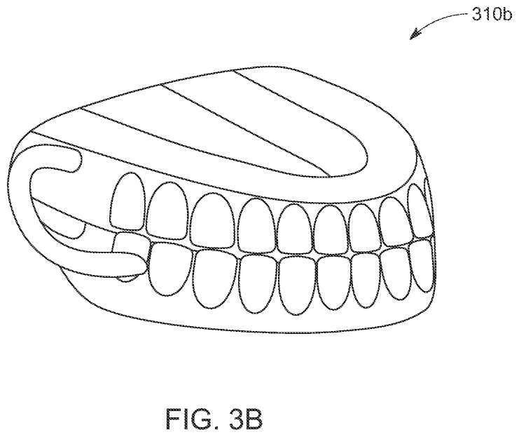

FIGS. 3A, 3B (collectively FIG. 3) show a prior art mouth guard 310a,b for protecting teeth. Mouth guard 310a shows the mouth guard with the dental structure 320 open while mouth guard 3b shows the mouth guard with the dental structure closed.

The mouth guard 310a,b has a first mouth guard section 320, a second mouth guard section 350, and a hinge section 390. The first mouth guard section 320 includes a first surface 322, a second surface (the surface against the upper teeth), an anterior region 326 and a posterior region 328. The first mouth guard section is adapted to receive the top teeth of a subject. The second mouth guard section 350 includes a first surface 352, a second surface (the surface against the bottom teeth), an anterior region 356 and a posterior region 358. The second mouth guard section is adapted to receive the bottom teeth of a subject. The hinge section 390 includes a superior end 392 and an inferior end 394. The superior end 392 of the hinge section 390 is connected to the posterior region 328 of the first mouth guard and the interior end 394 of the hinge section 390 is connected to the posterior region 358 of the second mouth guard 350. The hinge section is adapted to allow the anterior region of the first mouth guard section and the anterior region of the second mouth guard to open and close and the posterior region of the first mouth guard section and the posterior region of the second mouth guard to have limited movement with respect to each other.

The hinge section 390 is depicted on one side of the mouth guard in FIG. 3 with a similar opposing hinge on the other side of the mouth guard not shown. The hinge section 390 may take on any configuration that performs the above described function. The design and configuration of hinge sections for use with a mouth guard is well known in the art and a matter of design choice.

Mouth guard 310a,b is made from elastic material like soft dense plastic like Ethylene-vinyl acetate EVA. The materials for use in making mouth guards are well known and a matter of design choice.

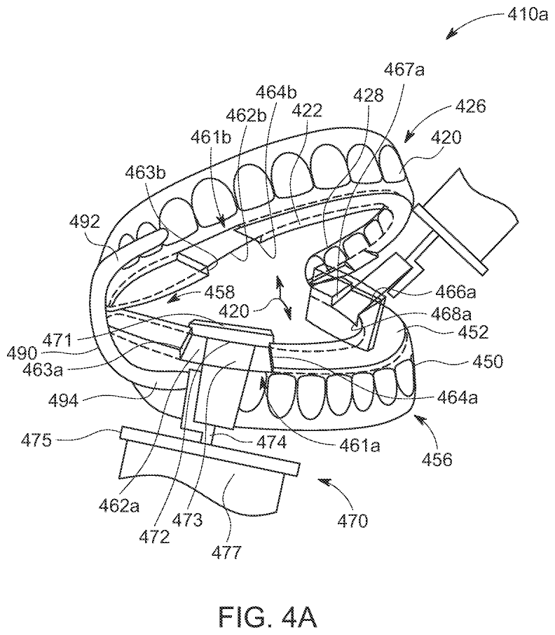

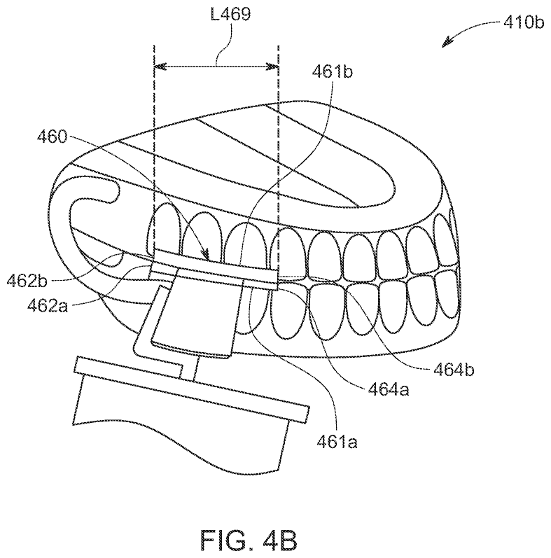

FIGS. 4A, 4B (collectively FIG. 4) show an illustrative embodiment bite tray 410a,b for alleviating dental discomfort of this disclosure configured for digital radiograph. Bite tray 410a shows the bite tray with the dental structure open while bite tray 4b shows the bite tray with the dental structure closed.

A bite tray 410a,b for alleviating dental discomfort includes a first mouth guard section 420, a second mouth guard section 450, a hinge section 490 (with similar opposing hinge section on the other side of the first and second mouth guards not shown), and a channel 460 (better shown in FIG. 4B). The first mouth guard section 420 includes a first surface 422, a second surface (the surface against the upper teeth), an anterior region 426 and a posterior region 428. The first mouth guard section 420 is adapted to receive the top teeth of a subject. The second mouth guard section 450 includes a first surface 452, a second surface (the surface against the bottom teeth), an anterior region 456 and a posterior region 458. The second mouth guard section 450 is adapted to receive the bottom teeth of a subject. The hinge section 490 includes a superior end 492 and an inferior end 494. The superior end 492 of the hinge section 490 is connected to the posterior region 428 of the first mouth guard and the interior end 494 of the hinge section 490 is connected to the posterior region 458 of the second mouth guard 450. The hinge section is adapted to allow the anterior region of the first mouth guard section and the anterior region of the second mouth guard to open and close and the posterior region of the first mouth guard section and the posterior region of the second mouth guard to have limited movement with respect to each other.

The channel 460 (best shown in FIG. 4B) is formed when the first surface 422 of the first mouth guard section 420 and the first surface 452 of the second mouth guard section 450 are brought together. The channel 460 extends along a predetermined length 469 of the first surface 422 of the first mouth guard section 420 and the first surface 452 of the second mouth guard section 450. The channel 460 opens on one side toward the facial direction of a subject and on another side toward the lingual direction of a subject. The channel is adapted to receive and hold a bite plate 472 of an intra-oral radiographic device 470.

In the illustrative example of FIG. 4, the channel 460 is formed by the coaction of sidewalls 462a. 463a. 464a of recess 461a in the first surface 452 of the second mouth guard section 450 and sidewalls 462b. 463b. 464b of recess 461b in the first surface 422 of the first mouth guard section 420.

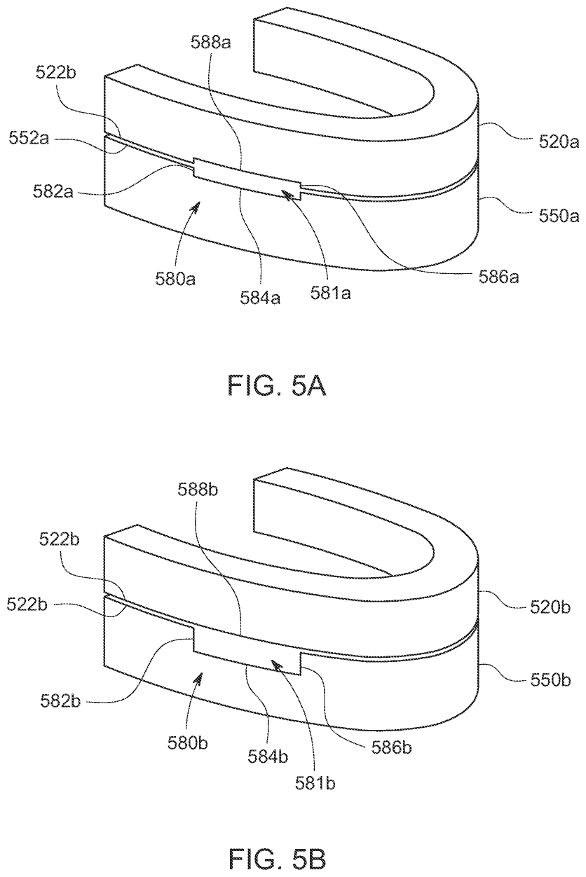

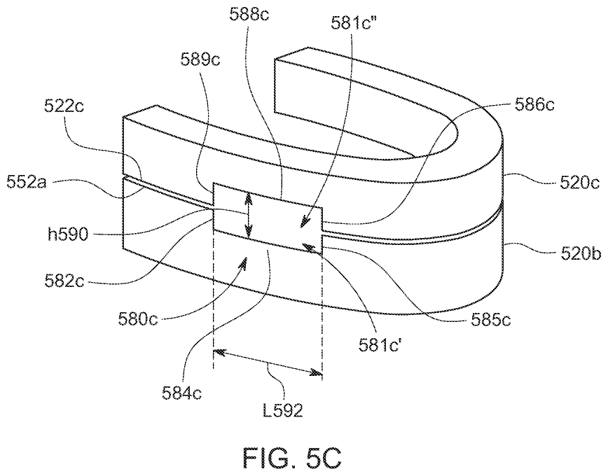

FIGS. 5A, 5B, 5C (collectively FIG. 5) show illustrative embodiments of channels formed in a bite tray according to this disclosure.

In the illustrative example of FIG. 5A, the channel 580a is formed by the coaction of sidewalls 582a, 584a, 586a of recess 581a in the first surface 552a of the second mouth guard section 550a and sidewalls 582a. 586a, 588a of recess 581a in the first surface 522 of the first mouth guard section 520a.

In the illustrative example of FIG. 5B, the channel 580b is formed by the coaction of sidewalls 582b, 584b, 586b of recess 581b in the first surface 552b of the second mouth guard section 550b and sidewall 588b of recess 581b in the first surface 522b of the first mouth guard section 520b.

In the illustrative example of FIG. 5C, the channel 580c is formed by the coaction of sidewalls 582c, 584c, 585c of recess 581c′ in the first surface 552c of the second mouth guard section 550c and sidewalls 586c, 588c, 589c of recess 581c″ in the first surface 522c of the first mouth guard section 520c.

The foregoing examples are illustrative only and there are other configurations that may be used for the channel. For example, the opposing walls of the channel may be parallel to each other or not. The specific shape and size of the channel is a design parameter that may be configured to receive and hold the intra-oral radiographic device. FIG. 5C also depicts the height 590 and the length 592 of the channel. The length 592 is the predetermined length that the channel extends along the first surface of the first mouth guard section and the first surface of the second mouth guard. The height 590 is the predetermined height of the channel extends between the first surface of the first mouth guard section and the first surface of the second mouth guard. The length 592 and height 590 are a design parameter that may be configured to receive and hold the intra-oral radiographic device.

In addition, any number of channels may be employed in accordance with this disclosure depending upon the intra-oral radiograph the bite tray of this disclosure is to be used for. For example there may be channels along both sides of the bite tray as well as a channel along the anterior portion of the bite tray with each channel configured to receive and hold the intra-oral radiographic device of choice.

Referring back to FIG. 4A, the bite tray 410a may be used in combination with an intra-oral radiograph 470. In the illustrative embodiment of FIG. 4A, the intra-oral radiograph is a digital radiograph comprising an image receptor 471 held by an image receptor support and holder 472, a bite block 473, a locator ring 475, a metal arm 474 and an x-ray source 477. The image receptor 471 is placed into the mouth of a patient. The receptor is positioned so as to lie parallel to the bite-platform. The patient bites on the bite block 473 attached to the image receptor 471 to hold the image receptor firmly against upper and lower teeth. The dental practitioner points the X-ray beam-aiming device 477 in the direction of the image receptor to expose the image receptor to X-ray radiation. The locator ring 475 and metal arm 473 are used by the practitioner as an aiming device to aim the X-ray. The image receptor captures the visible part of the upper and lower teeth as well as part of the roots and supporting bone.

The foregoing combination together form a system for intra-oral radiography. The system includes the bite tray 410a and the bite plate 473 and the image receptor 471. The previously described channel 460 of the bite tray is adapted to receive and hold the bite plate of the intra-oral radiographic device 470. The image receptor 471 is held against one or more teeth of either or both a left or right teeth set of a dental structure when the channel of the bite tray receives and holds the wing of the bitewing of the bitewing radiograph.

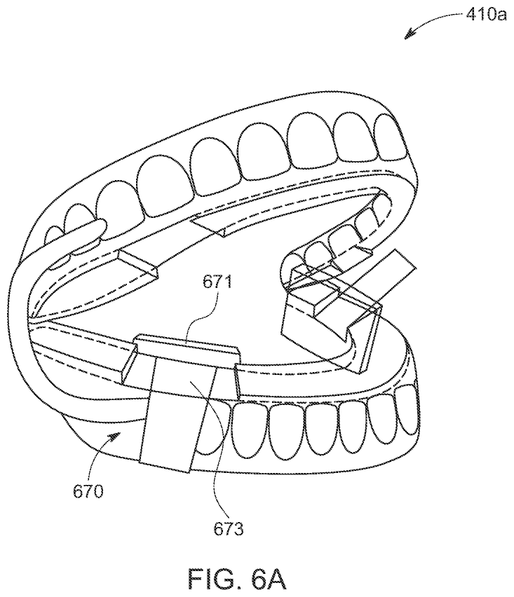

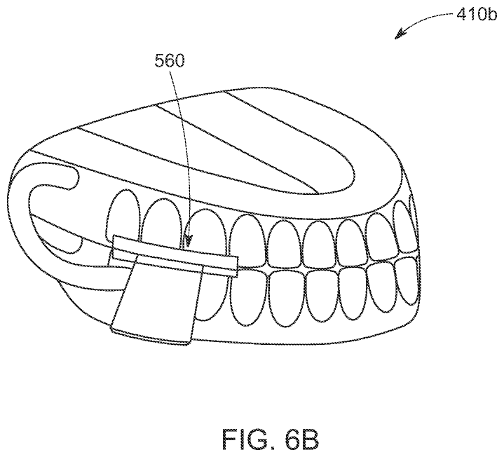

In the system depicted in FIG. 4, the intra-oral device is a digital radiograph. The system may be used with other intra-oral devices in a similar way. For example, the system could be used with a bitewing radiograph as depicted in FIG. 6. FIG. 6 depicts the bite tray 410a,b as that bite tray has been previously disclosed in FIG. 4 used with a bitewing radiograph 670 comprising an intraoral film packet 671 and a wing 673. The intraoral film packet is placed into the mouth of a patient. The image receptor is positioned so as to lie parallel to the bite-platform. The patient bites on the small wing attached to the intraoral film packet to hold the image receptor firmly against upper and lower teeth. The dental practitioner points an X-ray beam-aiming device in the direction of the image receptor to expose the image receptor to X-ray radiation. The image receptor captures the visible part of the upper and lower teeth as well as part of the roots and supporting bone.

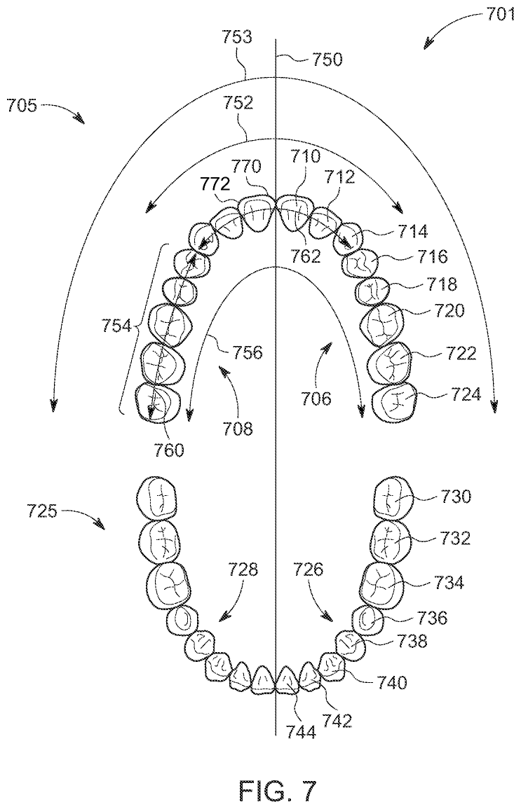

FIG. 7 depicts a dental anatomy for this disclosure. The dental structure 701 includes upper teeth 705 and lower teeth 725. The upper teeth 705 includes a right teeth set 706 and a left teeth set 708. The right teeth set 706 of the upper teeth 705 typically includes central incisor 710, lateral incisor 712, canine (cuspid) 714, first premolar (first bicuspid) 716, second premolar (second bicuspid) 718, first molar 720, second molar 722, and third molar (wisdom tooth) 724. These same teeth set also make up the left teeth set 708 albeit these teeth have not been called out in this discussion by number.

The lower teeth 725 includes a right teeth set 726 and a left teeth set 728. The right teeth set 726 of the lower teeth 725 typically includes central incisor 744, lateral incisor 742, canine (cuspid) 740, first premolar (first bicuspid) 738, second premolar (second bicuspid) 736, first molar 734, second molar 732, and third molar (wisdom tooth) 730. These same teeth set also make up the right teeth set 608 albeit these teeth have not been called out in this discussion by number.

When identifying teeth and referring to specific areas of a tooth, it is necessary to utilize named surfaces and directions designated according to where it is located. Teeth also have number/letter designations. There are several types of designation/identification systems, but the most common is the Universal System where the permanent dentition is numbered from 1-32 beginning with the maxillary right third molar.

FIG. 7 depicts these surfaces as follows. Distal 772 is the surface that is away from the midline 750 of the face. Facial 753 is the surface that faces the cheeks or lips. Labial 752 is the surface towards the lips. Buccal 754 is the surface towards the cheeks. Incisal 762 is the biting edge of an anterior tooth. Lingual 756 is the surface that faces the tongue. Mesial 770 is the surface that is closest to the midline of the face. Occlusal is the chewing surface of posterior teeth.

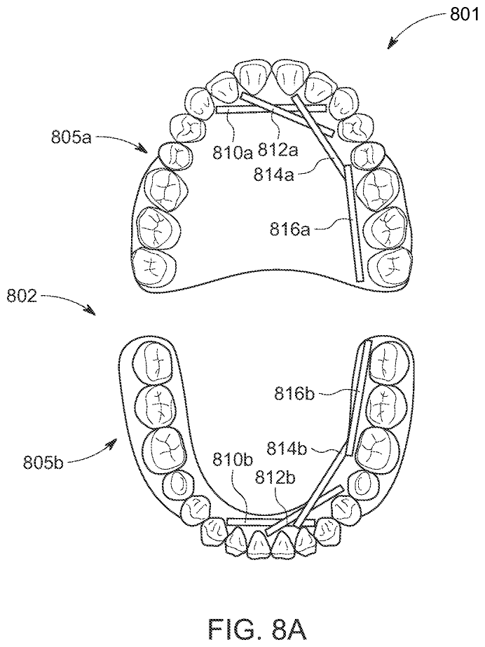

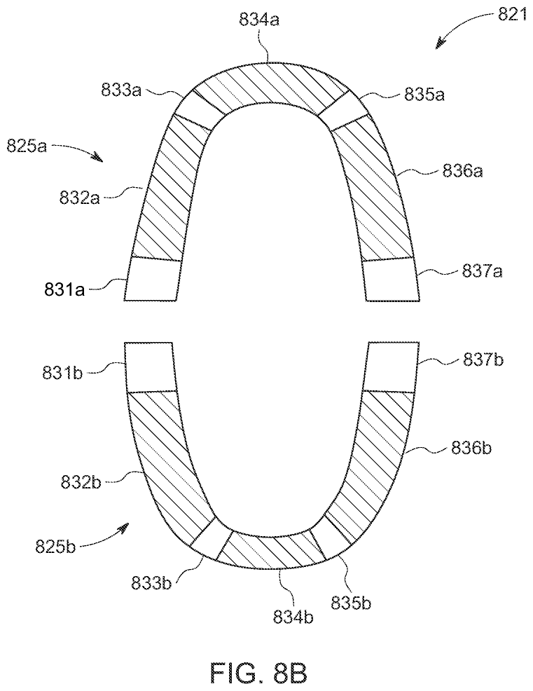

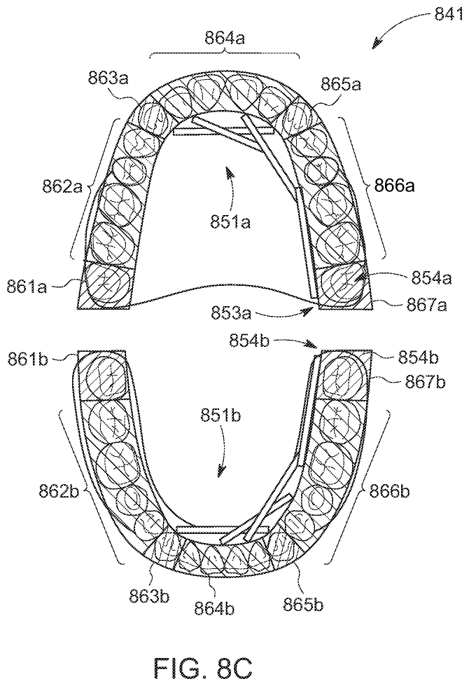

FIGS. 8A, 8B, 8C (collectively FIG. 8) show an illustrative embodiment of a bite tray according to this disclosure for bitewing radiograph. FIG. 8A depicts the dental structure 802 including upper teeth 805a and lower teeth 805b against which are placed intra-oral radiograph imaging device depicted as elements 810a, 812a, 814a, 816a in the picture of the upper teeth and as elements 810b, 812b, 814b, 816b in the picture of the lower teeth. FIG. 8B depicts an illustrative bite tray of this disclosure comprising channels formed when sidewalls of recesses 832a, 834a, 836a defined in first mouth guard section along the upper teeth and sidewalls of recesses 832b, 834b, 836b defined in the second mouth guard section along the lower teeth coact when the first surface of the first mouth guard section and the first surface of the second mouth guard are brought together as previously explained. FIG. 8C depicts the bite tray 853a,b overlaying teeth 854a,b with channels of this disclosure formed when sidewalls of recess 862a, 864a, 866a defined in first mouth guard section along the upper teeth and sidewalls of recess 862b, 864b, 866b defined in the second mouth guard section along the lower teeth coact when the first surface of the first mouth guard section and the first surface of the second mouth guard are brought together as previously explained.

By imaging device is meant an intraoral film packet of a bitewing radiograph, a sensor of a digital radiograph, an image capture device, and so on.

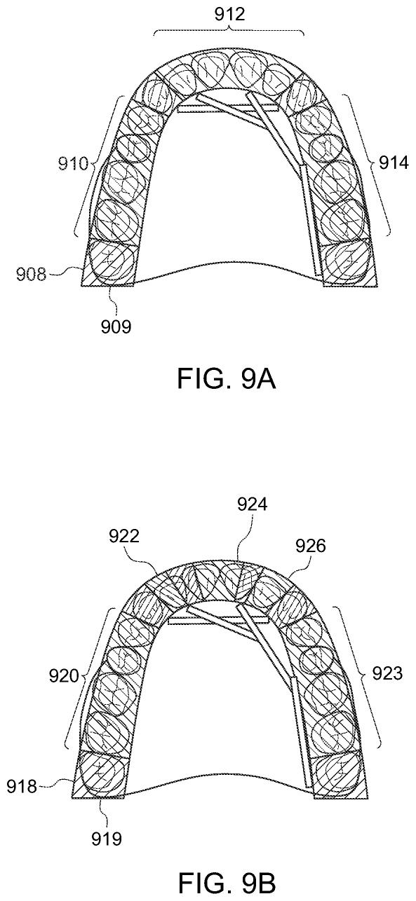

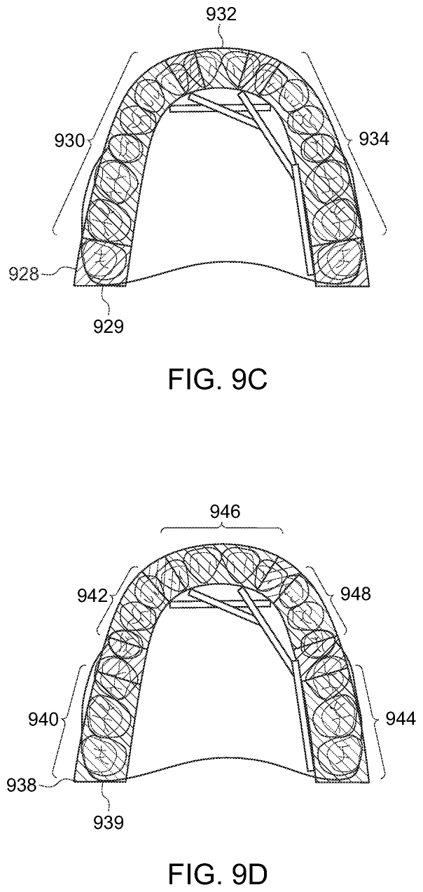

FIGS. 9A, 9B, 9C, 9D (collectively FIG. 9) show an illustrative embodiment of a bite tray according to this disclosure for bitewing radiograph. FIG. 9 depicts the bite tray 908, 918, 928, 938 overlaying teeth 909, 919, 929, 939 respectively with channels formed as previously explained. FIG. 9A depicts channels 910, 912, 914. FIG. 9B depicts channels 920, 922, 924, 926. FIG. 9C depicts channels 930, 932, 934. FIG. 9D depicts channels 940, 942, 946, 948, 949.

FIG. 9 discloses that a channel of this disclosure may be defined in a bite tray of this disclosure in a number of different configurations depending upon the intra-oral radiograph used and required to be received and held by the channel.

For example, a channel may extend along a right central incisor and right lateral incisor and a left central incisor and left lateral incisor of a dental structure as depicted by channel 912 in FIG. 9A. As another example, a channel may extend along a first bicuspid, a second bicuspid, a first molar, and a second molar of either or both a left or right teeth set of a dental structure as depicted by channel 910, 914 in FIG. 9A and channel 920, 923 in FIG. 9B.

As another example, a channel may extend along a right central incisor and left central incisor of a dental structure as depicted by channel 924 in FIG. 9B. As another example, a channel may extend along a lateral incisor as depicted by channels 922, 926 in FIG. 9B.

As another example, a channel may extend along a canine, a first bicuspid, a second bicuspid, a first molar, and a second molar of either or both a left or right teeth set of a dental structure as depicted by channel 930, 934 in FIG. 9C. FIG. 9C also shows it may also extend along the lateral incisor.

As another example, a channel may extend along a central incisor as depicted by channel 932 in FIG. 9B.

As another example, a channel may extend along a first molar and a second molar as depicted by channels 940, 944 in FIG. 9D. FIG. 9D also shows it may also extend along the third molar.

As another example, a channel may extend along a cuspid, a first bicuspid, and a second bicuspid as depicted by channels 942, 948 in FIG. 9D.

These examples explained as illustrative examples and other examples depicted in FIG. 9 are illustrative of channels that may be used with the bite tray of this disclosure. Any channel covering any one or more teeth of either or both side of either or both upper and lower mouth guard sections configured for use with the intra-oral radiograph of choice is within the scope of this disclosure.

As used herein, the term predetermined length means the length of a channel as explained above or another length desirable for a construct of a bite tray of this disclosure for use with an intra-oral radiograph of choice. The term predetermined height means the height of a channel as explained above or another height desirable for a construct of a bite tray of this disclosure for use with an intra-oral radiograph of choice.

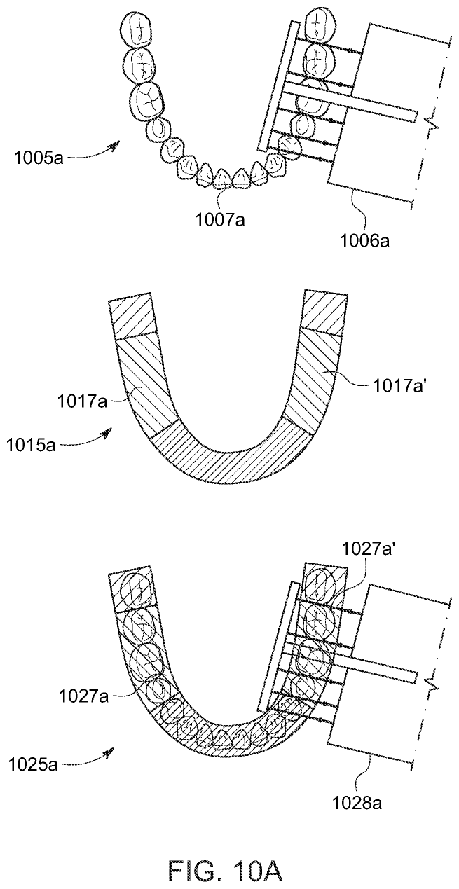

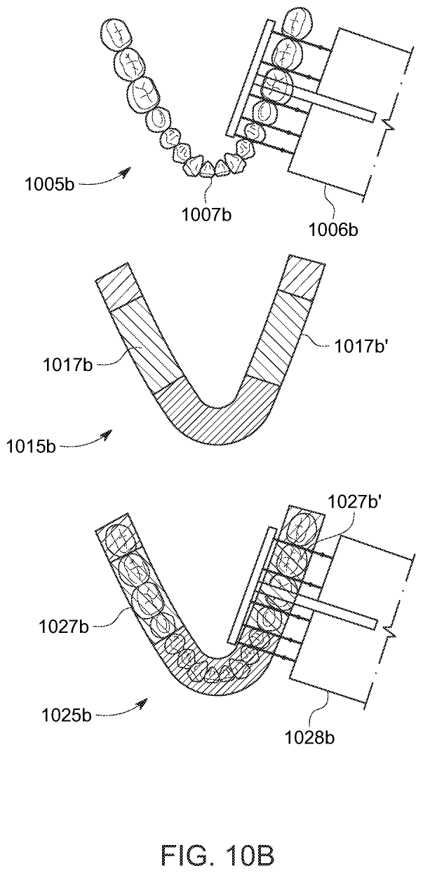

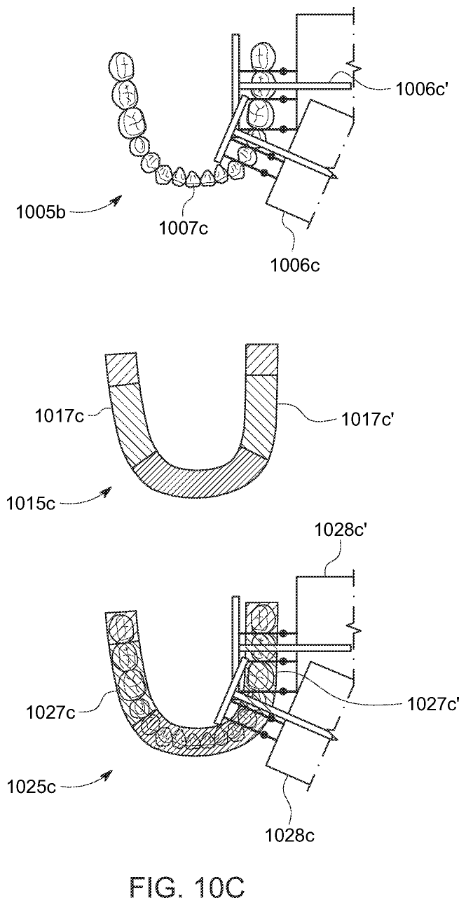

FIGS. 10A, 10B, 10C (collectively FIG. 10) show an illustrative embodiment of a bite tray according to this disclosure for digital radiograph. FIG. 10A shows illustrative disclosure for an Arch-shaped dental structure, FIG. 10B for a V-shaped dental structure, and FIG. 10C for a U-shaped dental structure. The teeth structure of each are depicted as elements 1005a,b,c respectively. The digital radiograph for each is depicted as elements 1006a,b,c respectively.

The bite plate 1015a,b,c for the respective teeth shape above are shown with channels 1017a,a′, 1017b,b′, 1017c,c′ respectively.

The overlay of the foregoing bite plates over teeth are depicted as elements 1025a, 1025b, 1025c with the recesses 1027a,a′, 1027b,b′, and 1027c,c′ respectively forming the respective channels when the first surface of the first mouth guard section and the first surface of the second mouth guard are brought together as previously explained.

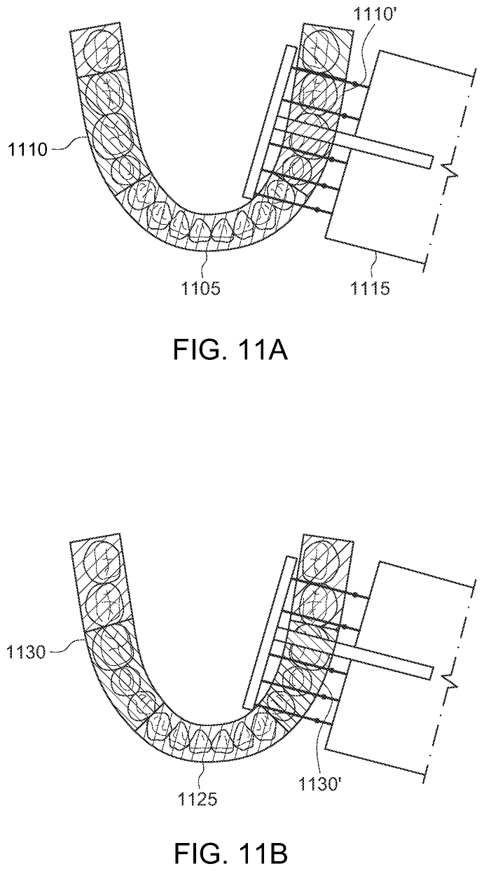

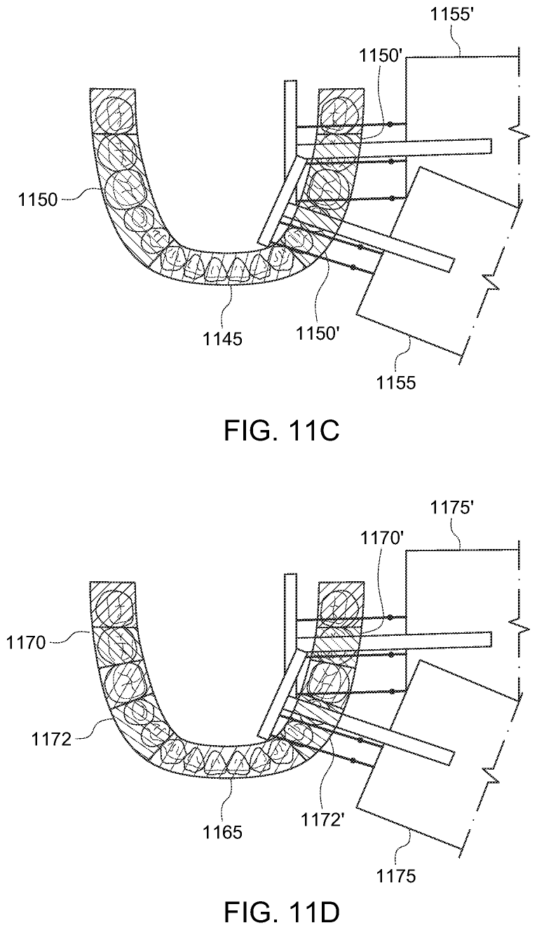

FIGS. 11A, 11B, 11C, 11D (collectively FIG. 11) show an illustrative embodiment of a bite tray according to this disclosure for digital radiograph. FIG. 11AB show illustrative disclosure for an Arch-shaped dental structure, FIG. 11C,D for a U-shaped dental structure. The bite plate 1105, 1125, 1145, 1165 for the respective teeth shape above are shown with recesses 1110, 1110′ shown in FIG. 11A, recesses 1130, 1130′ shown in FIG. 11B, recesses 1150, 1150′ shown in FIG. 11C, and recesses 1170, 1170′ and 1172, 1172′ shown in FIG. 11D.

The overlay of the foregoing bite plate over teeth with the recesses therein forming the respective channels when the first surface of the first mouth guard section and the first surface of the second mouth guard are brought together as previously explained.

These examples explained as illustrative examples and other examples depicted in FIG. 11 and throughout this disclosure are illustrative of channels that may be used with the bite tray of this disclosure. Any channel covering any one or more teeth of either or both side of either or both upper and lower mouth guard sections configured for use with the intra-oral radiograph of choice is within the scope of this disclosure. Illustrative intra-oral radiographs include the bite wing radiograph and the digital radiograph as previously explained.

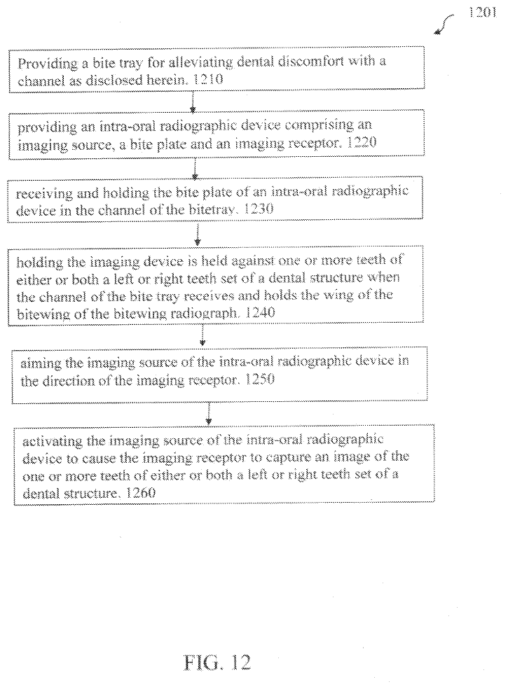

FIG. 12 shows a method for intra-oral radiography according to this disclosure. The method for intra-oral radiography includes the steps of: (1) providing a bite tray 1210 for alleviating dental discomfort. The bite tray includes a first mouth guard section, a second mouth guard section, a hinge section, and a channel. The first mouth guard section includes a first surface, a second surface, an anterior region and a posterior region. The first mouth guard section is adapted to receive the top teeth of a subject. The second mouth guard section includes a first surface, a second surface, an anterior region and a posterior region. The second mouth guard section is adapted to receive the bottom teeth of a subject.

The hinge section includes a superior end and an inferior end. The superior end of the hinge section is connected to the posterior region of the first mouth guard and the interior end of the hinge section connected to the second mouth guard. The hinge section is adapted to allow the anterior region of the first mouth guard section and the anterior region of the second mouth guard to open and close and the posterior region of the first mouth guard section and the posterior region of the second mouth guard to have limited movement with respect to each other.

The channel is formed when the first surface of the first mouth guard section and the first surface of the second mouth guard are brought together, the channel extending along a predetermined length of the first surface of the first mouth guard section and the first surface of the second mouth guard, the channel opening on one side toward the facial direction of a subject and on another side toward the lingual direction of a subject, the channel adapted to receive and hold a bite plate of an intra-oral radiographic device; (2) providing an intra-oral radiographic device 1220 including an imaging source, a bite plate and an imaging receptor; (3) receiving and holding 1230 the bite plate of an intra-oral radiographic device in the channel of the bite tray; (4) holding the imaging device 1240 against one or more teeth of either or both a left or right teeth set of a dental structure when the channel of the bite tray receives and holds the wing of the bitewing of the bitewing radiograph; (5) aiming the imaging source 1250 of the intra-oral radiographic device in the direction of the imaging receptor; and (6) activating the imaging source 1260 of the intra-oral radiographic device to cause the imaging receptor to capture an image of the one or more teeth of either or both a left or right teeth set of a dental structure.

In another aspect of the method, the bite plate of the system is a bitewing and the intra-oral radiographic device is a bitewing radiograph.

In another aspect of the method, the bite plate of the system is a bite block and the intra-oral radiographic device is a digital radiograph.

There is thus disclosed a bite tray for alleviating dental discomfort includes a first mouth guard section, a second mouth guard section, a hinge section, and a channel. The first mouth guard section includes a first surface, a second surface, an anterior region and a posterior region. The first mouth guard section is adapted to receive the top teeth of a subject. The second mouth guard section includes a first surface, a second surface, an anterior region and a posterior region. The second mouth guard section is adapted to receive the bottom teeth of a subject. The hinge section includes a superior end and an inferior end. The superior end of the hinge section is connected to the posterior region of the first mouth guard and the interior end of the hinge section is connected to the second mouth guard. The hinge section is adapted to allow the anterior region of the first mouth guard section and the anterior region of the second mouth guard to open and close and the posterior region of the first mouth guard section and the posterior region of the second mouth guard to have limited movement with respect to each other. The channel is formed when the first surface of the first mouth guard section and the first surface of the second mouth guard are brought together. The channel extends along a predetermined length of the first surface of the first mouth guard section and the first surface of the second mouth guard. The channel opens on one side toward the facial direction of a subject and on another side toward the lingual direction of a subject. The channel is adapted to receive and hold a bite plate of an intra-oral radiographic device.

In another aspect, the first surface of the first mouth guard includes a recess. The recess extends along a predetermined length of the first surface of the first mouth guard section. The recess forms the channel when the first surface of the first mouth guard section and the first surface of the second mouth guard are brought together.

In another aspect, the first surface of the second mouth guard includes a recess. The recess extends along a predetermined length of the first surface of the second mouth guard section. The recess forms the channel when the first surface of the first mouth guard section and the first surface of the second mouth guard are brought together.

In another aspect, the first surface of the first mouth guard includes a first recess. The first recess extends along a predetermined length of the first surface of the first mouth guard section. The first surface of the second mouth guard includes a second recess. The second recess extends along a predetermined length of the first surface of the second mouth guard section. The first recess of the first mouth guard and the second recess of the second mouth guard form the channel when the first surface of the first mouth guard section and the first surface of the second mouth guard are brought together.

In another aspect, the bite plate is a wing of a bitewing and the intra-oral radiographic device is a bitewing radiograph.

In another aspect, the bite plate is a bite block and the intra-oral radiographic device is a digital radiograph.

In another aspect, the bite tray may be used in combination with a bitewing radiograph including an intraoral film packet. The intraoral film packet is held against one or more teeth of either or both a left or right teeth set of a dental structure when the channel receives and holds the wing of the bitewing of the bitewing radiograph.

In another aspect, the bite tray may be used in combination with a digital radiograph including a sensor. The sensor is held against one or more teeth of either or both a left or right teeth set of a dental structure when the channel receives and holds the bite block of the digital radiograph.

In another aspect, the channel of the bite tray extends along a right central incisor and right lateral incisor and a left central incisor and left lateral incisor of a dental structure. In another aspect, the channel of the bite tray extends along a right central incisor and left central incisor of a dental structure.

In another aspect, the channel of the bite tray extends along a first bicuspid, a second bicuspid, a first molar, and a second molar of either or both a left or right teeth set of a dental structure.

In another aspect, the channel of the bite tray extends along a first cuspid, a first bicuspid, a second bicuspid, a first molar, and a second molar of either or both a left or right teeth set of a dental structure.

In another aspect, the channel of the bite tray extends along a first molar and a second molar of either or both a left or right teeth set of a dental structure.

In another aspect, the channel of the bite tray extends along a lateral incisor, a cuspid, a first bicuspid, a second bicuspid, a first molar, and a second molar of either or both a left or right teeth set of a dental structure.

In another aspect, the channel of the bite tray extends along a lateral incisor, a cuspid, a first bicuspid of either or both a left or right teeth set of a dental structure.

In another aspect, the channel of the bite tray extends along a first molar, and a second molar of either or both a left or right teeth set of a dental structure.

In another aspect, the channel of the bite tray extends along a first bicuspid and a second bicuspid of either or both a left or right teeth set of a dental structure.

In another aspect, the channel of the bite tray extends along a first molar of either or both a left or right teeth set of a dental structure.

In another aspect, the channel of the bite tray extends along a central incisor.

In another aspect, the channel of the bite tray extends along a lateral incisor of either or both a left or right teeth set of a dental structure.

In another aspect, the channel of the bite tray extends along a cuspid, a first bicuspid of either or both a left or right teeth set of a dental structure.

In another aspect, the channel of the bite tray extends along a lateral incisor, a cuspid of either or both a left or right teeth set of a dental structure.

In another aspect, the channel of the bite tray extends along a first bicuspid of either or both a left or right teeth set of a dental structure.

In another aspect, the channel of the bite tray extends along a second bicuspid of either or both a left or right teeth set of a dental structure.

In another aspect, the channel of the bite tray extends along a second molar of either or both a left or right teeth set of a dental structure.

In another aspect, the channel of the bite tray extends along a third molar of either or both a left or right teeth set of a dental structure.

In another aspect, a system for intra-oral radiography includes a bite tray for alleviating dental discomfort and an intra-oral radiographic device. The bite tray includes a first mouth guard section, a second mouth guard section, a hinge section, and a channel. The first mouth guard section includes a first surface, a second surface, an anterior region and a posterior region. The first mouth guard section is adapted to receive the top teeth of a subject. The second mouth guard section includes a first surface, a second surface, an anterior region and a posterior region. The second mouth guard section is adapted to receive the bottom teeth of a subject. The hinge section includes a superior end and an inferior end. The superior end of the hinge section is connected to the posterior region of the first mouth guard and the interior end of the hinge section connected to the second mouth guard. The hinge section is adapted to allow the anterior region of the first mouth guard section and the anterior region of the second mouth guard to open and close and the posterior region of the first mouth guard section and the posterior region of the second mouth guard to have limited movement with respect to each other. The channel is formed when the first surface of the first mouth guard section and the first surface of the second mouth guard are brought together, the channel extending along a predetermined length of the first surface of the first mouth guard section and the first surface of the second mouth guard, the channel opening on one side toward the facial direction of a subject and on another side toward the lingual direction of a subject. The intra-oral radiographic device includes a bite plate and an imaging device. The channel of the bite tray is adapted to receive and hold the bite plate of an intra-oral radiographic device. The imaging device is held against one or more teeth of either or both a left or right teeth set of a dental structure when the channel of the bite tray receives and holds the wing of the bitewing of the bitewing radiograph.

In another aspect of the system, the bite plate of the system is a bitewing and the intra-oral radiographic device is a bitewing radiograph.

In another aspect of the system, the bite plate of the system is a bite block and the intra-oral radiographic device is a digital radiograph.

In another aspect, a method for intra-oral radiography includes the steps of: (1) providing a bite tray for alleviating dental discomfort. The bite tray includes a first mouth guard section, a second mouth guard section, a hinge section, and a channel. The first mouth guard section includes a first surface, a first surface, an anterior region and a posterior region. The first mouth guard section is adapted to receive the top teeth of a subject. The second mouth guard section includes a first surface, a second surface, an anterior region and a posterior region. The second mouth guard section adapted to receive the bottom teeth of a subject. The hinge section includes a superior end and an inferior end. The superior end of the hinge section is connected to the posterior region of the first mouth guard and the interior end of the hinge section connected to the second mouth guard. The hinge section is adapted to allow the anterior region of the first mouth guard section and the anterior region of the second mouth guard to open and close and the posterior region of the first mouth guard section and the posterior region of the second mouth guard to have limited movement with respect to each other. The channel is formed when the first surface of the first mouth guard section and the first surface of the second mouth guard are brought together, the channel extending along a predetermined length of the first surface of the first mouth guard section and the first surface of the second mouth guard, the channel opening on one side toward the facial direction of a subject and on another side toward the lingual direction of a subject, the channel adapted to receive and hold a bite plate of an intra-oral radiographic device; (2) providing an intra-oral radiographic device including an imaging source, a bite plate and an imaging receptor; (3) receiving and holding the bite plate of an intra-oral radiographic device in the channel of the bite tray; (4) holding the imaging device against one or more teeth of either or both a left or right teeth set of a dental structure when the channel of the bite tray receives and holds the biteplate of the intra-oral radiographic device; (5) aiming the imaging source of the intra-oral radiographic device in the direction of the imaging receptor; and (6) activating the imaging source of the intra-oral radiographic device to cause the imaging receptor to capture an image of the one or more teeth of either or both a left or right teeth set of a dental structure.

In another aspect of the method, the bite plate of the system is a bitewing and the intra-oral radiographic device is a bitewing radiograph.

In another aspect of the method, the bite plate of the system is a bite block and the intra-oral radiographic device is a digital radiograph.

The bite tray may be placed into, moved about and positioned within the oral cavity of a patient in a way that creates less discomfort to the patient. The bite tray receives and holds a bite plate of an intra-oral radiographic device.

The bite tray of this disclosure allows intra-oral exams to be given by dentists in a way that may cause less patient discomfort. Less patient discomfort may arise from placement of the intraoral film packet or image receptor into the mouth of the patient. For, example, in finding the right position the intraoral film packet or image receptor may no longer be pressed against the gums of the patient. Instead the intraoral film packet or image receptor is pressed against the bite tray which sits comfortably between the intraoral film packet or image receptor and the mouth. The edges of the intraoral film packet or image receptor no longer press against and scrape the roof of the mouth during positioning. Instead, the edges of the intraoral film packet or image receptor packet press against and scrape the bite plate which sits comfortably between the intraoral film packet or image receptor and the mouth. Once properly positioned, the intraoral film packet or image receptor may no longer continue to press against the gums and roof of the mouth of the patient while the X-rays are being taken. Instead, the intraoral film packet or image receptor press against the bite plate which sits comfortably between the intraoral film packet and the mouth. The bite plate of this disclosure addresses this need for an intra-oral exam device that may be placed into, moved about and positioned within the oral cavity of a patient in a way that creates less discomfort to the patient.

Any material that is biologically compatible for use in an intra-oral cavity and having physical properties suitable for providing the features of the bite plate of this disclosure may be used.

While the disclosure has been described in conjunction with specific embodiments, it is evident that numerous alternatives, modifications, and variations will be apparent to those skilled in the art within the spirit and scope of the disclosure described above.

We claim:

1. A bite tray for alleviating dental discomfort, the bite tray comprising
a first mouth guard section comprising a first surface, a second surface, an anterior region and a posterior region, the first mouth guard section adapted to receive the top teeth of a subject;
a second mouth guard section comprising a first surface, a second surface, an anterior region and a posterior region, the second mouth guard section adapted to receive the bottom teeth of a subject;
a hinge section comprising a superior end and an inferior end, the superior end of the hinge section connected to the posterior region of the first mouth guard and the interior end of the hinge section connected to the second mouth guard, the hinge section adapted to allow the anterior region of the first mouth guard section and the anterior region of the second mouth guard to open and close and the posterior region of the first mouth guard section and the posterior region of the second mouth guard to have limited movement with respect to each other;
a channel formed when the first surface of the first mouth guard section and the first surface of the second mouth guard are brought together, the channel extending along a predetermined length of the first surface of the first mouth guard section and the first surface of the second mouth guard, the channel opening on one side toward the facial direction of a subject and on another side toward the lingual direction of a subject, the channel adapted to receive and hold a bite plate of an intra-oral radiographic device.

2. The bite tray of claim 1 wherein the first surface of the first mouth guard comprises a recess, the recess extending along a predetermined length of the first surface of the first mouth guard section, the recess forming the channel when the first surface of the first mouth guard section and the first surface of the second mouth guard are brought together.

3. The bite tray of claim 1 wherein the first surface of the second mouth guard comprises a recess, the recess extending along a predetermined length of the first surface of the second mouth guard section, the recess forming the channel when the first surface of the first mouth guard section and the first surface of the second mouth guard are brought together.

4. The bite tray of claim 1:
wherein the first surface of the first mouth guard comprises a first recess, the first recess extending along a predetermined length of the first surface of the first mouth guard section;
wherein the first surface of the second mouth guard comprises a second recess, the second recess extending along a predetermined length of the first surface of the second mouth guard section; and
wherein the first recess of the first mouth guard and the second recess of the second mouth guard forming the channel when the first surface of the first mouth guard section and the first surface of the second mouth guard are brought together.

5. The bite tray of claim 1 wherein the bite plate is a wing of a bitewing and the intra-oral radiographic device is a bitewing radiograph.

6. The bite tray of claim 1 wherein the bite plate is a bite block and the intra-oral radiographic device is a digital radiograph.

7. The bite tray of claim 5 in combination with a bitewing radiograph comprising an intraoral film packet, wherein the intraoral film packet is held against one or more teeth of either or both a left or right teeth set of a dental structure when the channel receives and holds the wing of the bitewing of the bitewing radiograph.

8. The bite tray of claim 6 in combination with a digital radiograph comprising a sensor, wherein the sensor is held against one or more teeth of either or both a left or right teeth set of a dental structure when the channel receives and holds the bite block of the digital radiograph.

9. The bite tray of claim 1 wherein the channel extends along a right central incisor and right lateral incisor and a left central incisor and left lateral incisor of a dental structure.

10. The bite tray of claim 1 wherein the channel extends along a right central incisor and left central incisor of a dental structure.

11. The bite tray of claim 1 wherein the channel extends along a first bicuspid, a second bicuspid, a first molar, and a second molar of either or both a left or right teeth set of a dental structure.

12. The bite tray of claim 1 wherein the channel extends along a first cuspid, a first bicuspid, a second bicuspid, a first molar, and a second molar of either or both a left or right teeth set of a dental structure.

13. The bite tray of claim 1 wherein the channel extends along a first molar and a second molar of either or both a left or right teeth set of a dental structure.

14. The bite tray of claim 1 wherein the channel extends along a lateral incisor, a cuspid, a first bicuspid, a second bicuspid, a first molar, and a second molar of either or both a left or right teeth set of a dental structure.

15. The bite tray of claim 1 wherein the channel extends along a lateral incisor, a cuspid, a first bicuspid of either or both a left or right teeth set of a dental structure.

16. The bite tray of claim 1 wherein the channel extends along a central incisor.

17. The bite tray of claim 1 wherein the channel extends along (a) a lateral incisor (b) a first bicuspid and a second bicuspid, (c) a cuspid, a first bicuspid, (d) a lateral incisor, a cuspid, or (e) a first bicuspid or a second bicuspid of either or both a left or right teeth set of a dental structure.

18. The bite tray of claim 1 wherein the channel extends along a first molar and a second molar, or a first molar or a second molar or a third molar or any combination thereof of either or both a left or right teeth set of a dental structure.

19. A system for intra-oral radiography comprising:
a bite tray for alleviating dental discomfort, the bite tray comprising
a first mouth guard section comprising a first surface, a second surface, an anterior region and a posterior region, the first mouth guard section adapted to receive the top teeth of a subject;
a second mouth guard section comprising a first surface, a second surface, an anterior region and a posterior region, the second mouth guard section adapted to receive the bottom teeth of a subject;
a hinge section comprising a superior end and an inferior end, the superior end of the hinge section connected to the posterior region of the first mouth guard and the interior end of the hinge section connected to the second mouth guard, the hinge section adapted to allow the anterior region of the first mouth guard section and the anterior region of the second mouth guard to open and close and the posterior region of the first mouth guard section and the posterior region of the second mouth guard to have limited movement with respect to each other;
a channel formed when the first surface of the first mouth guard section and the first surface of the second mouth guard are brought together, the channel extending along a predetermined length of the first surface of the first mouth guard section and the first surface of the second mouth guard, the channel opening on one side toward the facial direction of a subject and on another side toward the lingual direction of a subject; and
an intra-oral radiographic device comprising a bite plate and an imaging device;
wherein the channel of the bite tray is adapted to receive and hold the bite plate of an intra-oral radiographic device; and
wherein the imaging device is held against one or more teeth of either or both a left or right teeth set of a dental structure when the channel of the bite tray receives and holds the wing of the biteplate of the intra-oral radiographic device.

20. A method for intra-oral radiography comprising steps of:
Providing a bite tray for alleviating dental discomfort, the bite tray comprising
a first mouth guard section comprising a first surface, a second surface, an anterior region and a posterior region, the first mouth guard section adapted to receive the top teeth of a subject;
a second mouth guard section comprising a first surface, a second surface, an anterior region and a posterior region, the second mouth guard section adapted to receive the bottom teeth of a subject;
a hinge section comprising a superior end and an inferior end, the superior end of the hinge section connected to the posterior region of the first mouth guard and the interior end of the hinge section connected to the second mouth guard, the hinge section adapted to allow the anterior region of the first mouth guard section and the anterior region of the second mouth guard to open and close and the posterior region of the first mouth guard section and the posterior region of the second mouth guard to have limited movement with respect to each other;
a channel formed when the first surface of the first mouth guard section and the first surface of the second mouth guard are brought together, the channel extending along a predetermined length of the first surface of the first mouth guard section and the first surface of the second mouth guard, the channel opening on one side toward the facial direction of a subject and on another side toward the lingual direction of a subject, the channel adapted to receive and hold a bite plate of an intra-oral radiographic device; and
providing an intra-oral radiographic device comprising an imaging source, a bite plate and an imaging receptor;
receiving and holding the bite plate of an intra-oral radiographic device in the channel of the bite tray;
holding the imaging device is held against one or more teeth of either or both a left or right teeth set of a dental structure when the channel of the bite tray receives and holds the receives and holds the biteplate of the intra-oral radiographic device; and
aiming the imaging source of the intra-oral radiographic device in the direction of the imaging receptor;
activating the imaging source of the intra-oral radiographic device to cause the imaging receptor to capture an image of the one or more teeth of either or both a left or right teeth set of a dental structure.
